(19) United States
(12) Patent Application Publication
(10) Pub. No.: US 2008/0243097 A1
(43) Pub. Date: Oct. 2, 2008

(54) Male incontinency device

(76) Inventor: Robert Joseph Goss, Silver Spring, MD (US)

Correspondence Address: JONATHAN E. GRANT, 2107 HOUNDS RUN PLACE, SILVER SPRING, MD, 20096, US

(21) Appl. No.: 11/730,421

(22) Filed: Apr. 2, 2007

(51) Int. Cl. A61F 5/44

(52) U.S. Cl. 604/349

(57) ABSTRACT

A male incontinency device for men is disclosed which is convenient, efficient, easy to wear, and which allows no leakage. The male incontinency device comprises a gasket having an elastomeric structure with a hole in the center which fits around the genitals, a gasket retainer which surrounds and secures the gasket, a modified pair of briefs to secure the retainer, and a urine collection bag having the ability to collect and store urine, wherein an opening of the urine collection bag is secured to and around one rim of the gasket retainer.

### BACKGROUND OF THE DISCLOSURE

1. Field of the Disclosure

The present disclosure relates to an incontinence device, and more specifically, to an apparatus that allows for the removal, emptying, and replacement of a urine bag without any leakage of the system. The device can be used by men who suffer from urinary incontinence, which can lead to frequent and uncontrolled urination.

2. Description of the Prior Art

As the U. S. population ages, urinary incontinence among men has become a major physiological and psychological problem. Incontinence may be caused by weakened muscles around the bladder, bladder and/or prostate cancer, and the respective therapies to remove the cancer, nerve damage, damage to the nerve during bowel surgery, diabetes, and many other reasons. Urinary incontinence leaves the individual feeling insecure, not knowing when urine will start flowing, and whether or not such flow will start when they are away from known surroundings where they do know where to find the closest restroom.

Thus, in order to function and be mobile, a sufferer of urinary incontinence needs an effective urine collection device that will allow the user to go about his life with confidence. Going back to the mid 1800's, scores of male incontinence devices have been invented and patented. Examples of the prior art are as follows:

U.S. Pat. No. 6,152,903 (Falconer) discloses a urine collection device, a band for retaining the device and a method for attaching a medical device to the wearer's body. A band has two tabs. Each tab has one end attached to the band and a free end for extending through a hole in the device and attaching to the band. The band with the device mounted to it can be retained on the wearer's body.

U.S. Pat. No. 6,635,038 (Scovel) discloses an incontinence device having a disposable urinary bag having a plurality of attachment means located on its outside surface for securement of the disposable urinary bag to the adjustable attachment belt. Housed within the disposable urinary bag is an absorbent material for urine absorption. The disposable urinary bag also has in its upper central region an elastic ring or seal that provides a barrier against urine when a penis is inserted into it. On the top portion of the elastic ring, a slit is provided in conjunction with a sealing tab with adhesive to enlarge or constrict the size of the elastic ring. The adjustable attachment belt is comprised of a portion that fits around the wearer's waist with a plurality of attachment means to secure the upper portion of the disposable urinary bag in conjunction with a plurality of leg straps to provide connection means from the adjustable attachment belt to the lower portion of the disposable urinary bag.

U.S. Pat. No. 6,565,545 (Frenche) discloses a male catheter incontinence device for removably fitting on an end of a user's penis. The device is releasably couplable to underwear having a waistband worn by the user. The male incontinence device includes a housing that has a front wall and a back wall defining an interior of the housing. The back wall of the housing has an access opening for providing access to the interior of the housing. In one embodiment of the invention, the end of a penis is inserted into the interior of the housing through the access opening. An intermediate wall is mounted in the interior of the housing dividing the interior into an upper compartment and a lower compartment. In one embodiment of the present invention, the end of the penis is positioned in the upper compartment such that urine flows from the penis into the lower compartment of the housing.

U.S. Pat. No. 6,007,524 (Schneider) discloses an improved incontinence fluid collection and disposal system consisting of a flexible fluid reservoir assembly mounted in an opening in a reusable brief is presented. The brief is either adjustable or one piece but in either case holds the fluid reservoir assembly snugly against the body to restrict fluid leakage. An adhesive layer, normally in the form of a gasket and affixed to a tube on an inlet end of the fluid reservoir assembly, can be used to hold the fluid reservoir in position against a layer in the brief. Additional gasket layer(s) in the form of other materials can also be utilized. The fluid reservoir assembly is, in its preferred embodiment, supplied with a gelatin agent to gelatinize the urine to make for easy removal and disposal. A vacuum can be applied to the urine collection system to assist in urine evacuation from the fluid reservoir assembly.

However, these and other devices have various drawbacks. None of the prior art is guaranteed to be leakproof. Some incontinence devices require belts, while others require the mechanism to be glued to the body. Physicians report that the currently available products include various absorbent pads and catheter devices which are ineffective or uncomfortable.

The aging male population continues to look for a dependable, simple, over the counter device which meets their needs.

### SUMMARY OF THE DISCLOSURE

The proposed device can be easily and confidently worn by men of all shapes and sizes, including those who are physically active or by those confined to a nursing home.

The device is light weight, nonrestrictive, and easy to install, and is compact enough to be unnoticeable when the user is dressed in trousers, shorts or a bathing suit.

The device is dependably leak proof, and is unaffected by the degree of activity of the user, and can be used in the standing, sitting, and prone positions. This device makes the operation of a motor vehicle more comfortable.

In one embodiment of the disclosure, the device is comprised of three basic components. These components include a soft, pliable, gasket and a gasket retainer which fit around the penis and scrotum, and a urine collection bag which is attached to the gasket retainer.

In another embodiment of the disclosure, a securing mechanism secures the opening of the urine collection bag around the gasket retainer.

In another embodiment of the disclosure, a jockey brief has an opening for securing the brief to the retainer.

In yet another embodiment of the disclosure, specifically for one who is confined to a prone position, the catch bag is attached with straps to the user's thigh, when the user is confined to a prone position.

### BRIEF DESCRIPTION OF THE DRAWINGS

Other objects and advantages of the invention will be evident from the following detailed description, with like reference numbers referring to items throughout.

FIG. 1 is a cross-sectional side view of the device in use;

FIG. 2 is a frontal view of gasket and gasket retainer assembly;

FIG. 3 is a cross section side view of the gasket and gasket retainer assembly;

FIG. 4 is a frontal view of the modified pair of briefs;

FIG. 5 is a perspective view of the catch bag and means of attachment;

FIG. 6 is a side view as to how to install the device;

FIG. 7 is a perspective view of an elongated bag for use in the prone position; and

FIG. 8 is a view of the hinged retainer.

### DETAILED DESCRIPTION OF THE DISCLOSURE

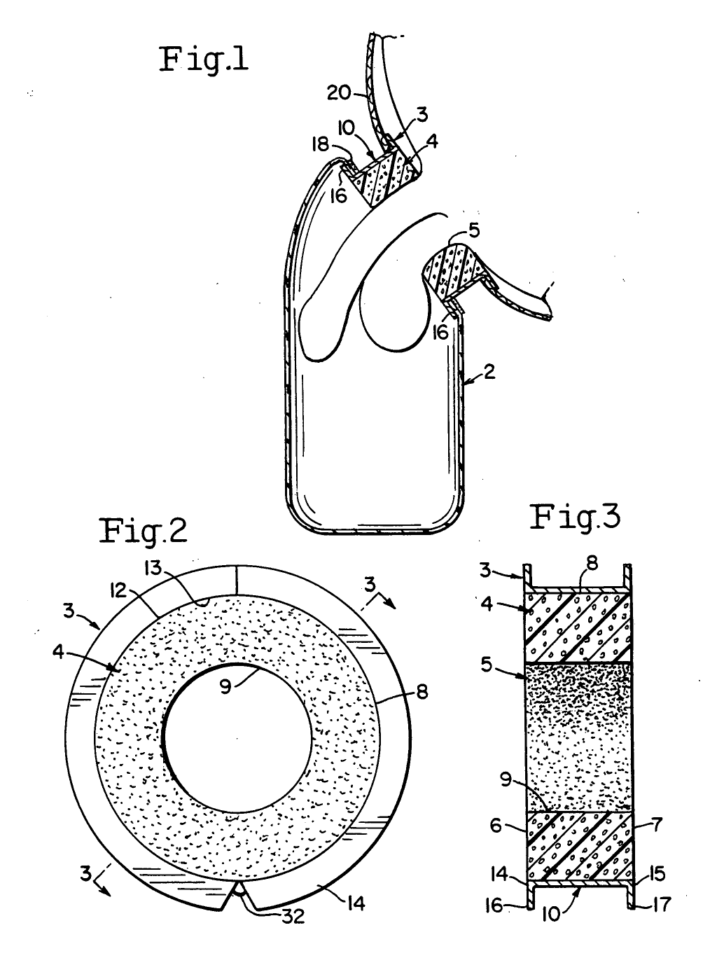

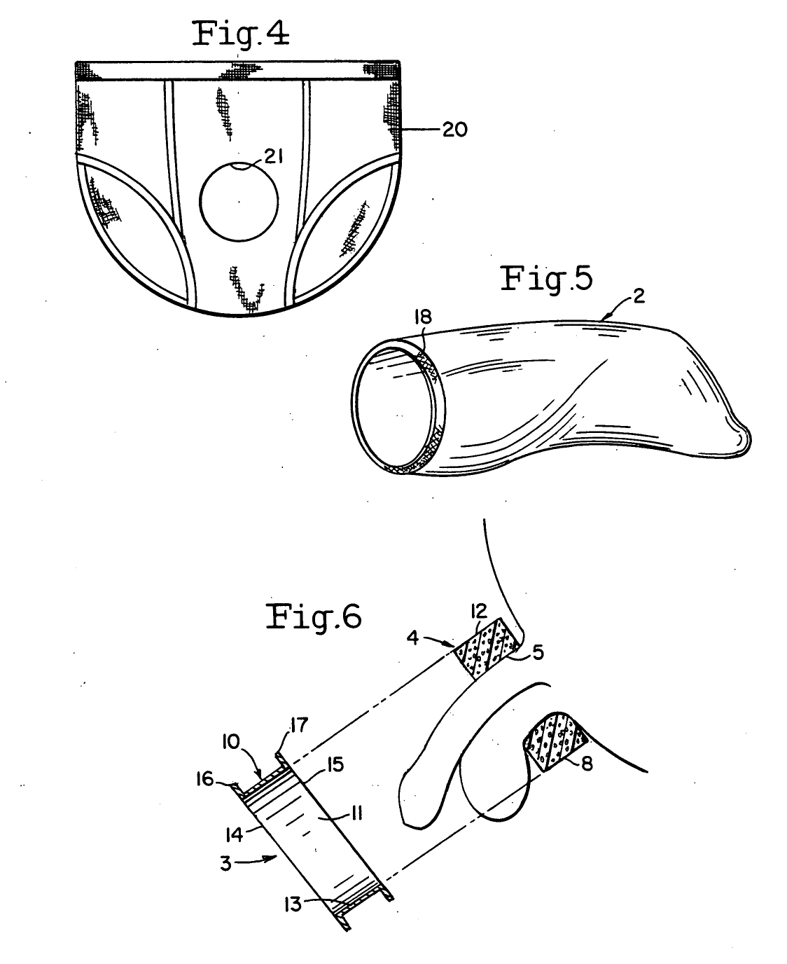

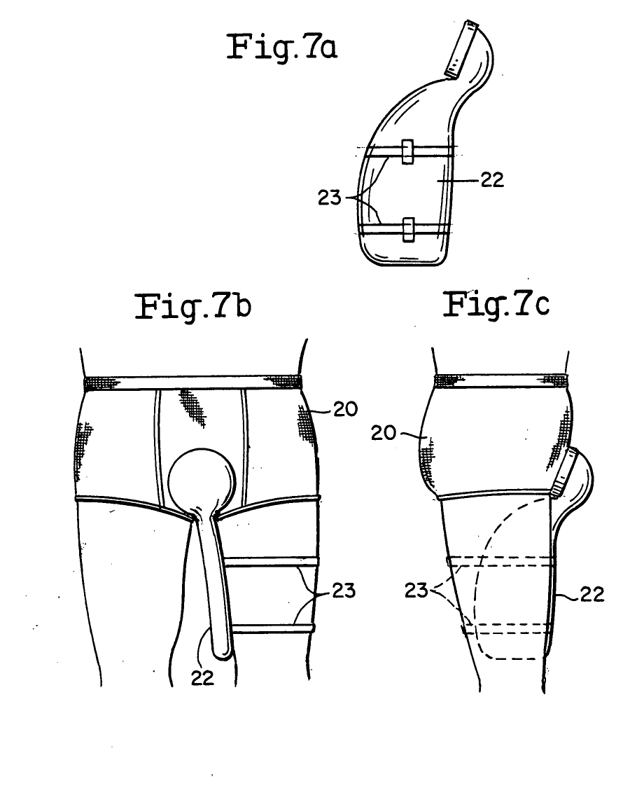

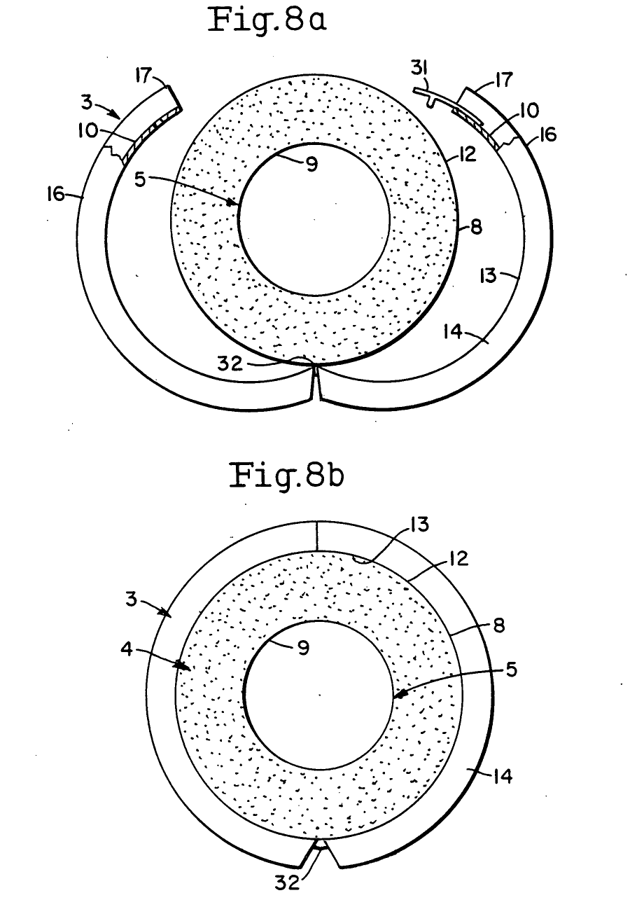

Referring to FIGS. 1-8, the device comprises a leak proof gasket 4 around the genitals, a gasket retainer 3, a jockey brief 20 and urine collection catch bag 2.

The gasket is a circular, donut shaped elastomeric device, with an opening 5, in its center, having memory so as to maintain its shape. It is preferable that the material used to make the gasket is a thermoplastic elastomer which allows the gasket to be easily stretched, but still has a stable configuration. Other suitable materials having the same or similar properties may be used.

It is most preferable that both sides 6 and 7 be flat. The circular outside circumferential surface 8 of the gasket 4 is flat and smooth. The choice of materials facilitates a non-slipping interaction of the parts.. The circular inside surface 9 of the gasket 4 is flat and smooth. Because the men using this device vary greatly in age, size, and weight, different sizes of the gasket and gasket retainer may be desired. Thus, the opening 5 of the circular gasket 4 may be of variable sizes.

The gasket is easily stretched so as to fit around the genitals (FIG. 1).

A gasket retainer 3 is a plastic hinged structure, which, when held together by a clamp or clasp 31, comprises a short tube 10, having an inside diameter 11 equal to or slightly less than that of the outside diameter 12 of the gasket, such that the gasket is held snugly in the gasket retainer. Thus the gasket cannot slip or fall out of said gasket retainer. The inside surface 13 of the gasket retainer 3 is in communication with the outside surface 12 of the gasket. A plastic or metal hinge 32 positioned on the gasket retainer opposite the clamp 31, allows the gasket retainer to open so that it can be positioned around the gasket. When in the open or unclamped position, the gasket retainer is comprised of the hinge 32 connecting two halves of the short tube 10.

Each of the sides 14 and 15 of the closed gasket retainer 3 has a lip 16 and 17. The urine collection catch bag 2 fits over the lip 16 that is distal from the wearer's body. The urine collection catch bag 2 is held in place by an elastic means which ensures a leak proof connection to the retainer. A pull string or elastic 18 may be positioned at or around the opening 19 of the bag 2. Alternatively, the urine collection catch bag can be held in place over the lip by means of an elastic o-ring. The urine collection catch bag can be provided in different lengths and milliliter capacity. The appropriate size of the urine collection bag is dependent upon the age, size and health of the individual using the urine collection device.

In an alternative embodiment, the gasket retainer 3 is a one piece structure, preferably plastic, with the structure preferably having a high degree of rigidity, and having the ability to fit over and around the gasket 4. The gasket retainer 3 comprises a short tube 10, having an inside diameter 11 equal to or slightly less than that of the outside diameter 12 of the gasket, such that the gasket is held snugly in the gasket retainer. Thus the gasket cannot slip or fall out of said gasket retainer. The inside wall 13 of the gasket retainer 3 is in communication with the outside wall 12 of the gasket.

The urine collection bag itself may be of any material that can hold a mildly acidic solution.

Preferably, a leakproof, plastic bag, strong enough to hold fluids, should be used.

When the gasket and gasket retainer are in place around the genitals, the brief 20 is drawn in place around the retainer as shown in FIG. 1. The modified pair of briefs 20 will have a mounting hole 21 positioned in the front of the briefs, wherein the mounting hole has the same diameter as that of the diameter of the tube of the retainer. The rim 17 proximal to the body of the user will fit behind the material surrounding the mounting hole 21 of the briefs 20. The catch bag is then secured to the retainer. The individual can then dress normally.

If a patient is immobile or bed ridden, in a prone position or lying on his back or side, it may be most advantageous and perhaps necessary to have an extended catch bag 22 which will be attached to the thigh with straps 23. The bag would be positioned preferably on the inner side of the leg. The catch bag may be modified to hold a greater amount of fluid. The plastic sheet of the bag will have a slightly heavier gage to withstand possible tension stresses not experienced in standard usage.

The user will likely consult with a physician or urologist to select the proper sized gasket.

Many modifications and variations of the present disclosure are possible in light of the above teachings. It is, therefore, to be understood within the scope of the appended claims the disclosure may be protected otherwise than as specifically described.

What is claimed is:

1) A male incontinency device, said urine collection device comprising:
a) a gasket, said gasket comprising an elastomeric structure with a hole in the center so as to fit male genitals said gasket;
b) a gasket retainer, said gasket retainer comprising:
i) a short tube;
ii) a front side;
iii) a back side;
iv) a lip around said front side; and
v) a second lip around said back side;
wherein said gasket retainer is sized to fit snugly around said gasket;
c) a urine collection bag, said urine collection bag having the ability to collect and store urine, wherein an opening of said urine collection bag has the ability to be secured to and around said lip around said front side of said gasket retainer; and
d) a modified pair of briefs to secure the gasket retainer.

2) The male incontinency device of claim 1, wherein the gasket is comprised of a material selected from the group consisting of a thermoplastic elastomer and other suitable material.

3) The male incontinency device of claim 1, wherein the gasket comprises a circular shaped structure.

4) The male incontinency device of claim 1, wherein said gasket retainer comprises a molded plastic structure.

5) The male incontinency device of claim 4, wherein said gasket retainer is rigid.

6) The male incontinency device of claim 1, wherein said gasket retainer further comprises a hinge and a clamp, such that said gasket retainer can be opened to fit around a gasket.

7) The male incontinency device of claim 5, wherein said gasket retainer fits snugly around said gasket.

8) The male incontinency device of claim 6, wherein said gasket retainer fits snugly around said gasket.

9) The male incontinency device of claim 1, wherein said urine collection bag further comprises an elastic positioned around the opening of said urine collection bag.

10) The male incontinency device of claim 1, wherein said urine collection bag further comprises a draw string positioned around the opening of said urine collection bag.

11) The male incontinency device of claim 1, further comprising an o-ring, wherein said o-ring secures the opening of said urine collection bag around said gasket retainer.
